(19) United States
(12) Patent Application Publication
(10) Pub. No.: US 2008/0027547 A1
(43) Pub. Date: Jan. 31, 2008

(54) Prosthetic device for spinal joint reconstruction

(75) Inventors: Kidong Yu, Memphis, TN (US); Eric Lange, Collierville, TN (US)

Correspondence Address: HAYNES AND BOONE, LLP, 901 Main Street, Suite 3100, Dallas, TX, 75202, US

(73) Assignee: Warsaw Orthopedic Inc., Warsaw, IN (US)

(21) Appl. No.: 11/494,311

(22) Filed: Jul. 27, 2006

(51) Int. Cl. A61F 2/44

(52) U.S. Cl. 623/17.13; 623/17.14

(57) ABSTRACT

A prosthetic device for placement in an intervertebral space defined between an upper vertebra and a lower vertebra to provide articulating motion to the upper and lower vertebrae. The prosthetic device may include a first articular portion configured to be at least partially disposed in the intervertebral space and adjacent to the first vertebra and a second articular portion configured to be at least partially disposed in the intervertebral space and adjacent to the second vertebra. A hemispherical member is also included, about which the first and second articular portions can articulate. A flexible member flexibly secures the hemispherical member to the first articular portion. A method of implanting also is disclosed.

### BACKGROUND

Disc arthroplasty is one way of treating injured, degraded, or diseased spinal joints. Some disc arthroplasty treatments include replacing injured discs of the joint with a motion-preserving spinal disc that allows some articulation or movement of the spinal joint. Often, these motion-preserving spinal discs are attached to the adjacent vertebra using screws as fasteners. By themselves, these fasteners can be undesirable for various reasons

In additional to preserving the motion of the spinal disc, it is often desired to preserve motion in another portion of the space between adjacent vertebra. For example, a facet joint between to vertebrae often requires replacement. A replacement for a facet joint needs to provide sufficient wear resistance and shock absorption.

What is needed is a prosthetic device for insertion into an intervertebral space that provides an advancement over the prior art. The posterior joint replacement device disclosed herein overcomes one or more problems in the prior art.

### SUMMARY

In one exemplary aspect, the present disclosure is directed to a prosthetic device for placement in an intervertebral space defined between an upper vertebra and a lower vertebra to provide articulating motion to the upper and lower vertebrae. The prosthetic device may include a first articular portion configured to be at least partially disposed in the intervertebral space and adjacent to the first vertebra and a second articular portion configured to be at least partially disposed in the intervertebral space and adjacent to the second vertebra. A hemispherical member is also included, about which the first and second articular portions can articulate. A flexible member flexibly secures the hemispherical member to the first articular portion.

In some embodiments, the first articular portion includes a first posterior section and a first interdiscal section. The first posterior section is connectable to an anterior arch of the first vertebra and the first posterior section is connectable to a posterior arch of the first vertebra.

In some embodiments, the second articular portion includes a second posterior section and a second interdiscal section. The second posterior section is connectable to an anterior arch of the second vertebra and the second posterior section is connectable to a posterior arch of the second vertebra.

In another exemplary aspect, the present disclosure is directed to a motion-preserving prosthetic device component for placement in an intervertebral space defined between a first vertebra and a second vertebra. The motion-preserving prosthetic device includes an intervertebral section configured to be at least partially disposed in the intervertebral space, and a posterior section configured to be at least partially disposed outside of the intervertebral space. The intervertebral section and the posterior section each separately support motion, and the posterior section includes a flexible movement controlling mechanism.

In another exemplary aspect, the present disclosure is directed to a method of implanting a prosthetic device. The prosthetic devices includes an interbody component, a posterior component, and a combined interbody/posterior component. The method includes placing the combined interbody/posterior component in an intervertebral space between first and second vertebrae, the combined interbody/posterior component being placed to engage with an anterior arch and a posterior arch of the first vertebra. The method also includes placing the interbody component in the intervertebral space, the interbody component being placed to engage with an anterior arch of the second vertebra. The method further includes placing the posterior component in the intervertebral space, the posterior component being placed to engage with a posterior arch of the second vertebra.

In some embodiments, the method further includes attaching the posterior component to the posterior arch of the second vertebra by introducing a fastener through an aperture in the posterior component.

In some exemplary aspects, the joint replacement device and method disclosed herein may include one or more features disclosed in the following prior patent applications, incorporated herein in their entirety by reference:

U.S. Utility patent application Ser. No. 11/343,159, filed on Jan. 30, 2006 and entitled “Prosthetic Device For Spinal Joint Reconstruction;”

U.S. Utility patent application Ser. No. 11/342,961 Jan. 30, 2006, filed on Jan. 30, 2006 and entitled “Prosthetic Device For Spinal Joint Reconstruction;”

U.S. Utility patent application Ser. No. 11/031,602, filed on Jan. 7, 2005 and entitled “Spinal Arthroplasty Device and Method;”

U.S. Utility patent application Ser. No. 11/031,603, filed on Jan. 7, 2005 and entitled “Dual Articulating Spinal Device and Method;”

U.S. Utility patent application Ser. No. 11/031,780, filed on Jan. 7, 2005 and entitled “Split Spinal Device and Method;”

U.S. Utility patent application Ser. No. 11/031,904, filed on Jan. 7, 2005 and entitled “Interconnected Spinal Device and Method;”

U.S. Utility patent application Ser. No. 11/031,700, filed on Jan. 7, 2005 and entitled “Support Structure Device and Method;”

U.S. Utility patent application Ser. No. 11/031,783, filed on Jan. 7, 2005 and entitled “Mobile Bearing Spinal Device and Method;”

U.S. Utility patent application Ser. No. 11/031,781, filed on Jan. 7, 2005 and entitled “Centrally Articulating Spinal Device and Method;” and

U.S. Utility patent application Ser. No. 11/031,903, filed on Jan. 7, 2005 and entitled “Posterior Spinal Device and Method.”

### BRIEF DESCRIPTION OF THE DRAWINGS

FIG. 1 is a pictorial representation of a lateral view of a portion of a vertebral column.

FIG. 2 is a pictorial representation of a lateral view of a pair of adjacent vertebrae bodies defining an intervertebral space.

FIG. 3 is a pictorial representation of top view of an exemplary intervertebral prosthetic device according to one or more embodiments of the present invention.

FIG. 4 is a pictorial representation of a top view of two of the prosthetic devices of FIG. 3 placed on one of the vertebrae of FIG. 2.

FIG. 5 is a pictorial representation of a lateral view of a pair of adjacent vertebrae with the prosthetic device of FIG. 3 placed there between.

FIG. 6 is a pictorial representation of an isometric view of an embodiment of a lower articular portion of the intervertebral prosthetic device of FIG. 3.

FIG. 7 is a pictorial representation of an isometric view of another embodiment of a lower articular portion of the intervertebral prosthetic device of FIG. 3.

FIG. 8 is a pictorial representation of an isometric view of an exemplary intervertebral prosthetic device according to another group of embodiments of the present invention.

FIG. 9 is a pictorial representation of an isometric view of an embodiment of a lower articular portion of the intervertebral prosthetic device of FIG. 8 with an upper articular portion shown in phantom.

FIG. 10 is a pictorial representation of an isometric view of an exemplary intervertebral prosthetic device according to another group of embodiments of the present invention.

FIG. 11 is a pictorial representation of an isometric view of an embodiment of a lower articular portion of the intervertebral prosthetic device of FIG. 10 with an upper articular portion shown in phantom.

FIG. 12 is a pictorial representation of a lateral view of an embodiment of the lower articular portion of the intervertebral prosthetic device of FIG. 10, being in a flexion position relative to the upper articular portion (shown in phantom).

FIG. 13 is a pictorial representation of a lateral view of an embodiment of the lower articular portion of the intervertebral prosthetic device of FIG. 10, being in an extension postition relative to the upper articular portion (shown in phantom).

FIG. 14 is a pictorial representation of an isometric view of an exemplary intervertebral prosthetic device according to another group of embodiments of the present invention, the intervertebral prosthetic device being in a flexion position.

FIG. 15 is a pictorial representation of an isometric view of the intervertebral prosthetic device of FIG. 14, the intervertebral prosthetic device being in an extension position.

FIG. 16 is a pictorial representation of an isometric view of the intervertebral prosthetic device of FIG. 14, the intervertebral prosthetic device being in a lateral tension (translated) position.

FIG. 17 is a pictorial representation of an isometric view of an exemplary intervertebral prosthetic device according to another group of embodiments of the present invention.

FIG. 18 is a pictorial representation of an isometric view of the intervertebral prosthetic device of FIG. 17, the intervertebral prosthetic device being in a lateral tension (translated) position.

FIG. 19 is a pictorial representation of an isometric view of an exemplary intervertebral prosthetic device according to another group of embodiments of the present invention.

FIG. 20 is a pictorial representation of an isometric view of the intervertebral prosthetic device of FIG. 19, the intervertebral prosthetic device being in a flexion position.

FIGS. 21-24 are pictorial representations of isometric views of an exemplary intervertebral prosthetic device according to another group of embodiments of the present invention.

FIG. 25 is a pictorial representation of a top view of two of the prosthetic devices placed on one of the vertebrae of FIG. 2.

### DETAILED DESCRIPTION

The present invention relates generally to vertebral reconstructive devices and, more particularly, to an intervertebral prosthetic device for implantation. For the purposes of promoting an understanding of the principles of the invention, reference will now be made to embodiments or examples illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. References numerals may be repeated throughout the embodiments, which does not by itself require that the item or feature identified by the reference numeral is the same for each embodiment, or is even thereby required. Moreover, the formation of a first feature over, next to, or on a second feature in the description that follows may include embodiments in which the first and second features are in direct contact, and may also include embodiments in which additional features may be formed interposing the first and second features, such that the first and second features may not be in direct contact.

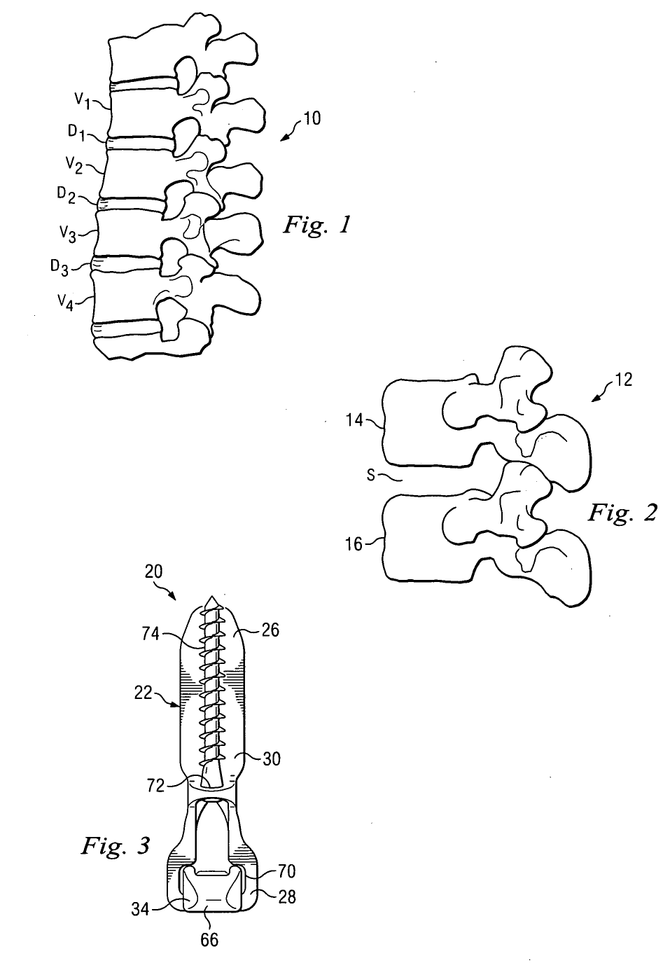

FIG. 1 shows a lateral view of a portion of a spinal column 10, illustrating a group of adjacent upper and lower vertebrae V1, V2, V3, V4 separated by natural intervertebral discs D1, D2, D3. The illustration of four vertebrae is only intended as an example. Another example would be a sacrum and one vertebra.

For the sake of further example, two of the vertebrae will be discussed with reference to FIG. 2. The two vertebrae form a spinal segment 12 including an upper vertebra 14 and a lower vertebra 16. Some types of disc arthroplasty require that some or all of the natural disc that would have been positioned between the two vertebrae 14, 16 be removed via a discectomy or a similar surgical procedure. Removal of the diseased or degenerated disc results in the formation of an intervertebral space S between anterior arches of the upper and lower vertebrae 14, 16. In addition, removal of any portion of the posterior arches of the upper and lower vertebrae 14, 16 may also be performed to increase the intervertebral space S in which a prosthesis can be implanted.

Although the illustration of FIG. 2 generally depicts the spinal segment 12 as a lumbar vertebral joint, it is understood that the devices, systems, and methods of this disclosure may also be applied to all regions of the vertebral column, including the cervical and thoracic regions. The present invention can be readily applied to various vertebrae, including vertebra that do not directly form the intervertebral space S, but that are at locations respectively above and below those vertebra that directly form the intervertebral space S.

Some conventional spinal prosthetic devices are installed using an anterior procedure, requiring a physician to access the spinal column using distressing and sometimes traumatic procedures. Once a prosthetic is installed using an anterior procedure, scar tissue may build on sensitive vessels. If a second procedure is required, a physician may be required to remove the scar tissue to access the previously placed prosthetic. This sensitive procedure can cause additional distress to the patient. The intervertebral prosthetic device disclosed herein may be advantageous over prior devices because it may be installed using a posterior procedure. Accordingly, a physician need not access and disturb the critical vessels that reside at the anterior side of the spinal column. Further, if a second procedure becomes necessary, the physician has easy access to the previously placed prosthetic without removing scar tissue off of sensitive vessels. Accordingly, the procedure may be simplified and may cause less distress to the patient.

The embodiments described below provide many benefits, some of which may include a decreased modulus in the overall design of the implant, improved wear resistance, improved posterior stress distribution, and/or less complex surgical requirements. Additional and/or different advantages for each of the embodiments described below will also be readily apparent.

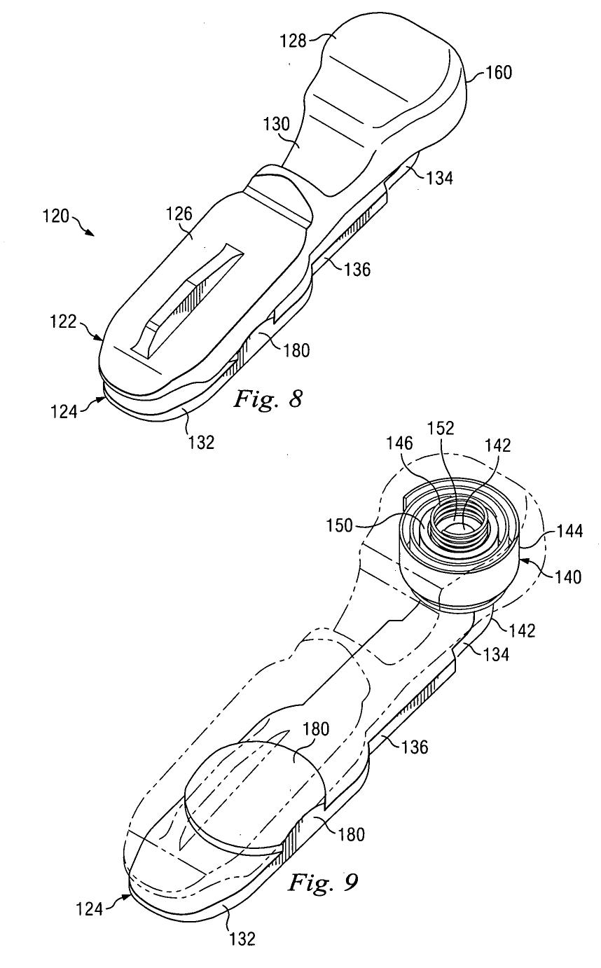

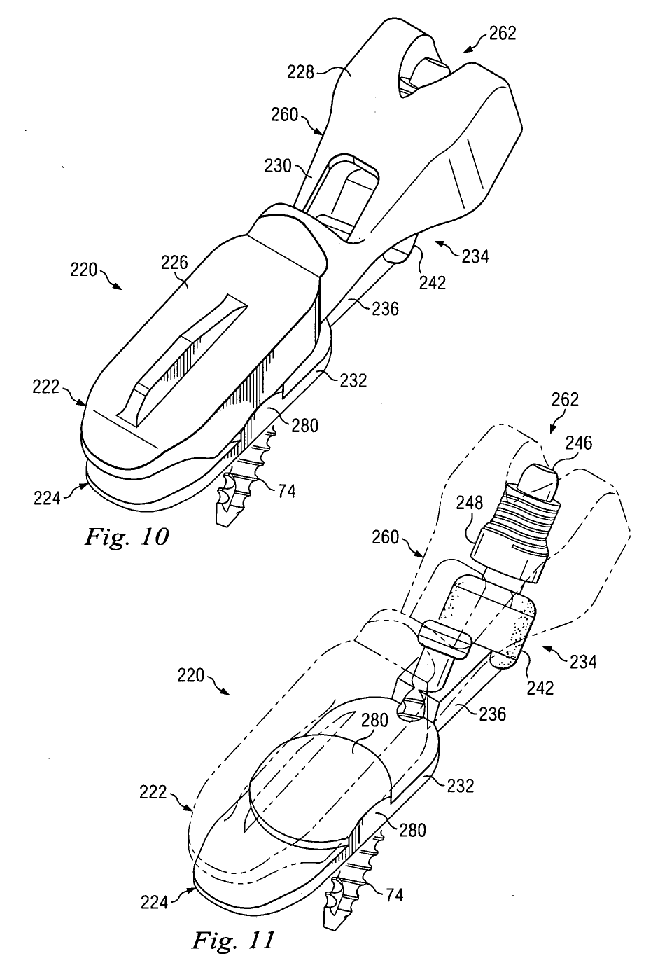

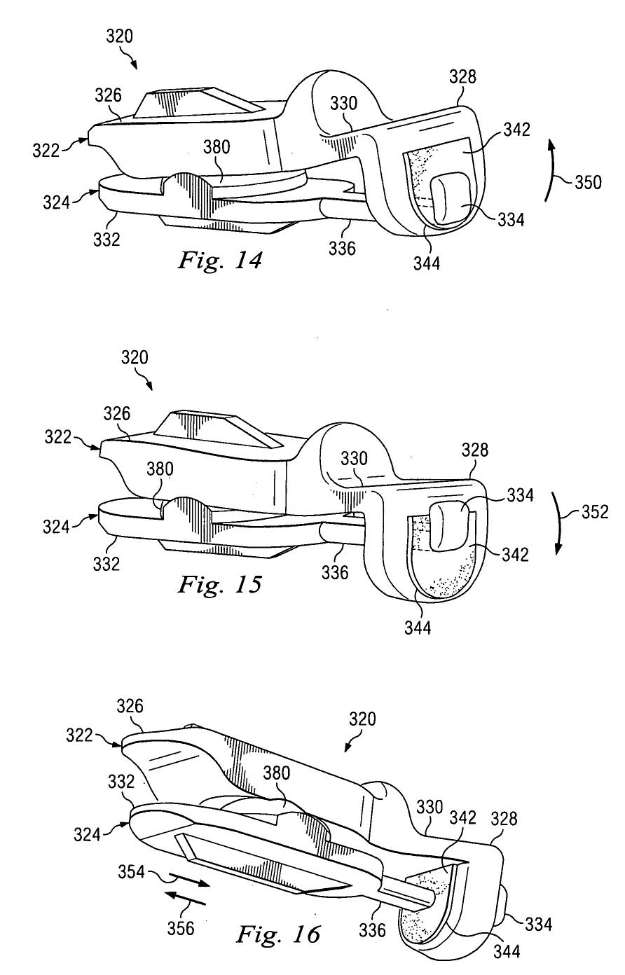

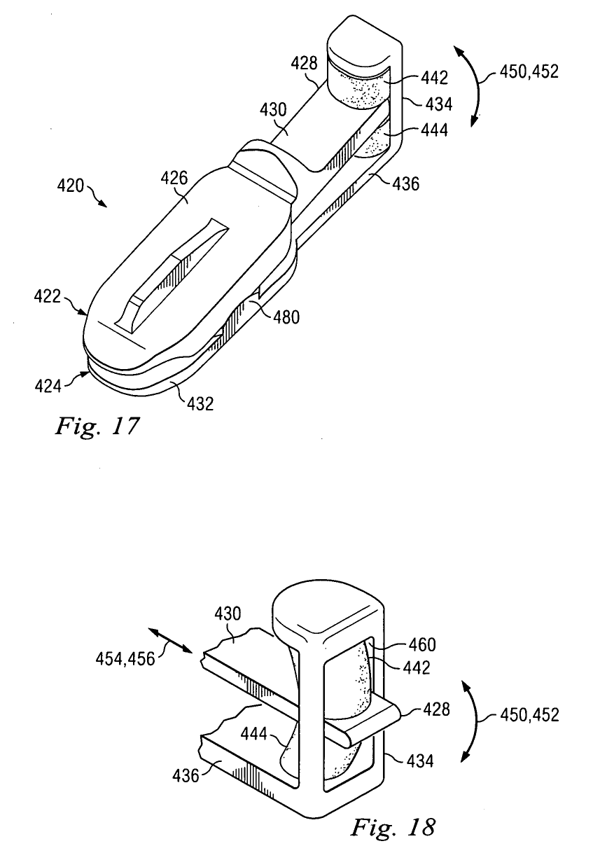

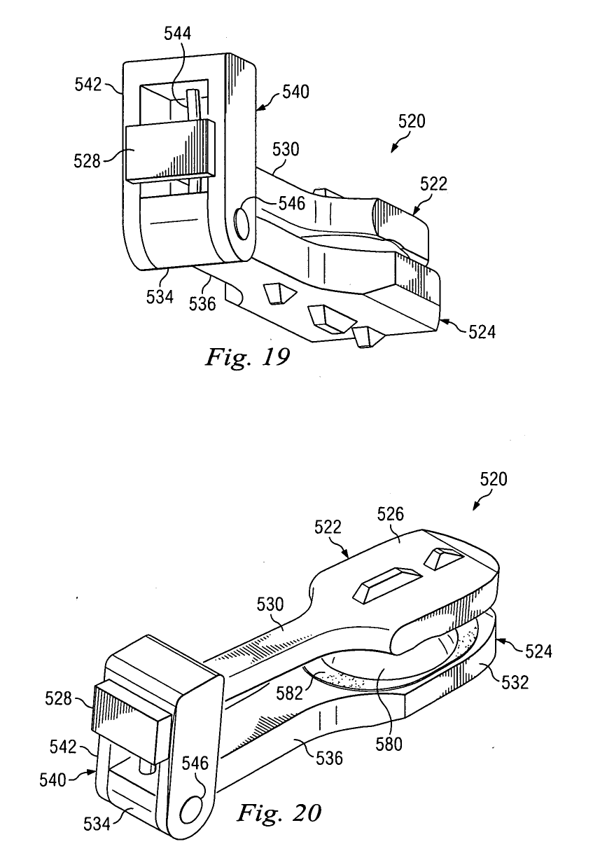

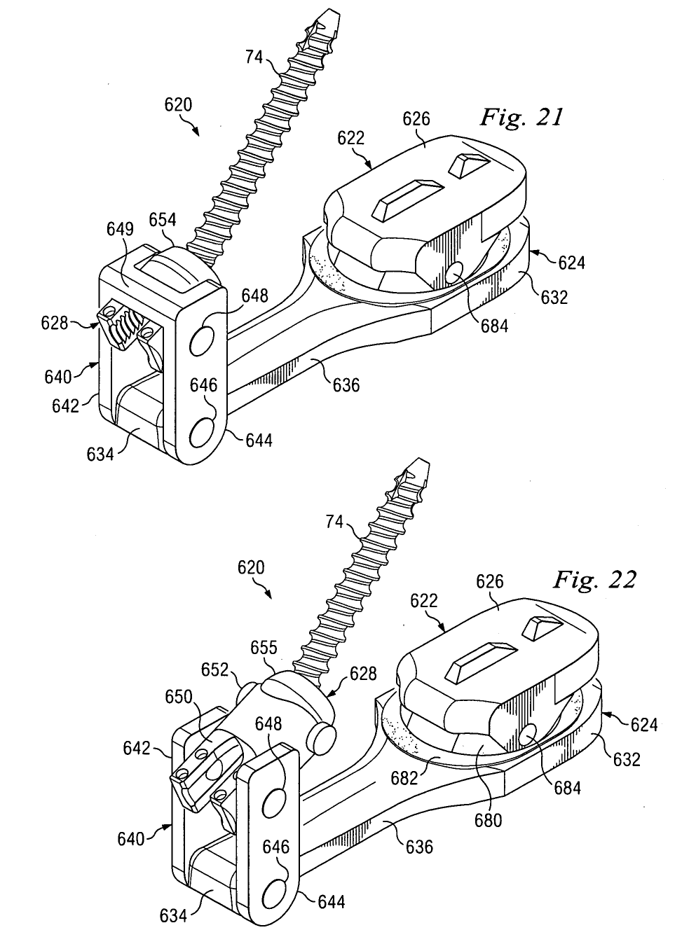

For the sake of general reference, FIGS. 3-7 show a first group of embodiments, identified as prosthetic device 20. FIGS. 8-9 show a second group of embodiments, identified as prosthetic device 120. FIGS. 10-13 show a third group of embodiments, identified as prosthetic device 220. FIGS. 14-16 show a fourth group of embodiments, identified as prosthetic device 320. FIGS. 17-18 show a fifth group of embodiments, identified as prosthetic device 420. FIGS. 19-20 show a sixth group of embodiments, identified as prosthetic device 520. FIGS. 21-24 show a seventh group of embodiments, identified as prosthetic device 620. Each of the prosthetic devices 20, 120, 220, 320, 420, 520, and 620 allows the vertebra 14 to articulate relative to the vertebra 16 to provide movement to the spinal joint. The disclosed prosthetic devices are sized to fit the intervertebral space height in a manner similar to a natural intervertebral disc, such as any of discs D1-D4. In some embodiments, the prosthetic devices are provided in pairs, although other embodiments may have different numbers of devices.

Referring to FIGS. 3-5, in one embodiment, the prosthetic device 20 includes an upper articular portion 22 and a lower articular portion 24. The upper articular portion 22 includes an upper main body formed of an interdiscal section 26, a posterior section 28, and a bridge 30 extending between the interdiscal and posterior sections 26, 28. Similarly, the lower articular portion 24 includes a lower main body formed of an interdiscal section 32, a posterior section 34, and a bridge 36 extending between the interdiscal and posterior sections 32, 34.

The upper and lower articular portions 22, 24 may be formed of any suitable biocompatible material including metals such as cobalt-chromium alloys, titanium alloys, nickel titanium alloys, and/or stainless steel alloys. Ceramic materials such as aluminum oxide or alumina, zirconium oxide or zirconia, compact of particulate diamond, and/or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. The various sections comprising the upper articular portion 22 and the lower articular portion 24 may be formed of different materials thus permitting metal on metal, metal on ceramic, metal on polymer, ceramic on ceramic, ceramic on polymer, or polymer on polymer constructions.

In the exemplary embodiment shown, each of the upper and lower articular portions 22, 24 are integrally formed or molded of a single piece of material. In other embodiments, one or more of the interdiscal, posterior, and bridge sections of either of the upper or lower articular portions 22, 24 may be formed separately and attached to one or more of the other sections. Attachments in these embodiments may be accomplished using any fastening mechanism known in the art including, for example, a threaded connection, a bolted connection, or a latched connection, among others. In those embodiments, the interdiscal, posterior, and bridge sections also may be formed of different materials.

The interdiscal section 26 of the upper articular portion 22 may include a bone contacting surface 38 and an inner surface 44 opposite the bone contacting surface 38. A first articular surface may form a part of the inner surface 44. In the embodiment shown, the first articular surface is a recess. Similarly, the interdiscal section 32 of the lower articular portion 24 may include a bone contacting surface 40 opposite an inner surface 48, with a second articular surface forming a part of the inner surface and being configured to mate with the first articular surface. In the embodiment shown, the second articular surface is a protrusion.

Together, the first and second articular surfaces may form an articulating joint that allows the upper and lower articular portions 22, 24 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere that may rotate or translate within the first articular surface, forming a loosely constrained ball and socket style joint. Although shown as a ball and socket joint, the first and second articular surfaces may be any shape or design that allows one of the upper and lower articular portions 22, 24 to move relative to the other of the upper and lower articular portions 22, 24. For example, the first and second articular surfaces may include a trough and recess, a ball and saucer, or other shaped features. In some embodiments, the first and second articular surfaces are formed of a material different than the remainder of the interdiscal sections 26, 32 to provide suitable articulation.

The bone contacting surfaces 38, 40 of the upper and lower articular portions 22, 24 may include features or coatings which enhance the fixation of the implanted prosthetic device 20. For example, the surfaces 38, 40 may be roughened such as by chemical etching, bead-blasting, sanding, grinding, serrating, and/or diamond-cutting. All or a portion of the bone contacting surfaces 38, 40 of the upper and lower articular portions 22, 24 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, and/or other surface textures and features.

In the exemplary embodiment shown, optional upper and lower bone connectors 50, 52 are formed on the bone contacting surfaces 38, 40, respectively. These bone connectors 50, 52 extend toward the upper and lower vertebrae 14, 16 in a manner to help secure the upper and lower articular portions 22, 24 in place. In the example shown, the bone connectors 50, 52 are keels configured to extend into notches or grooves formed into the vertebral endplates. The bone connectors also could be a series of ridges, protrusions, or other surface features that help fix the prosthetic device 20 in place.

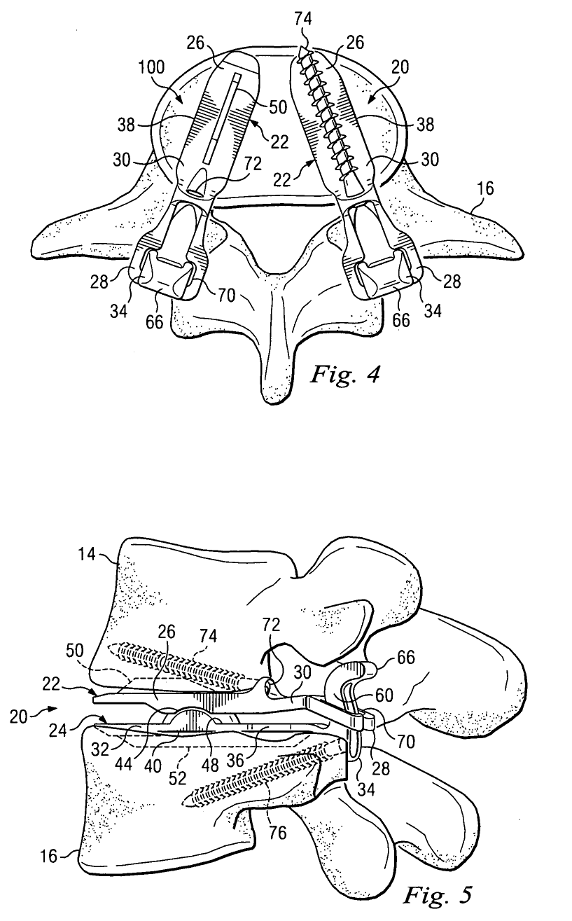

The bridge sections 30, 36 extend rearward from the interdiscal sections 26, 32 respectively. In the embodiment shown, the bridge sections 30, 36 extend substantially along a longitudinal centerline 58 (FIG. 4) of the prosthetic device 20. In other embodiments, the bridge sections do not align with a longitudinal centerline of the interdiscal sections, but may be curved or angled to depart away from the longitudinal centerline.

The posterior sections 28, 34 may be disposed at the end of the bridge sections 30, 36 and, in some embodiments, may be configured to fit adjacent to the processes (e.g., the articular spinous process of the facet joint) of the vertebrae 14, 16. The posterior section 34 of the lower articular portion 24 may include a tail 60 extending generally in a direction along the spinal column, and past the posterior section 28 of the upper articular portion 22.

The tail 60 may connect to the bridge section 36 and, in the example shown, is formed by a bend in the bridge section 36. Extending upwardly, the tail may be at least partially disposed at a location higher than the bridge section 36. Part of the tail may form a motion stop 66 configured to limit the range of articulation between the upper and lower articular portions 22, 24. In the embodiment shown, the motion stop 66 is a bend in the tail 60 having a length that is configured to work together with the upper articular portion 22 to limit the available range of articular rotation of the upper and lower articular portions 22, 24. It should be noted that the tail 60 may be substantially straight or may be curved, angled or otherwise formed. In one exemplary embodiment, the tail 60 may include a curve concentric with the curvature of the protruding articular surface 46.

The posterior section 28 of the upper articular portion 22 includes an aperture 70 formed therein that is configured to receive the tail 60 of the lower articular portion 24. In the embodiment shown, a portion of the posterior section 28 forms a motion stop that is configured to cooperate with the motion stop 66 on the tail 60. Accordingly, when the upper and lower articular portions 22, 24 are assembled as shown in FIG. 5, the motion stop 66 and the motion stop cooperate to limit the range of articulation of the prosthetic device 20. In addition, the aperture 70 is configured so that when the articulating surfaces 42, 46 are mated, the tail 60 extends through the aperture 70 in a manner that articulation may still freely occur within the range.

In the embodiment shown, the upper articular portion 22 includes an attachment element, such as a screw hole 72, extending upwardly from the upper main body of the upper articular portion 22 and a fastener 74. The screw hole 72 is configured to connect the fastener 74 to the upper main body, and thereby secure the upper articular portion 22 to the superior vertebrae 14. Similarly, a screw hole (described in greater detail below) and fastener 76 secure the upper articular portion 22 to the superior vertebrae 14.

The fasteners 74, 76 may be bone screws having a threaded portion for insertion into bone and a head operable to secure in the corresponding screw holes. The fasteners 74, 76 may be inserted into the bones substantially in a plane formed through the longitudinal axis, and in the embodiment shown, the fasteners are substantially parallel to the longitudinal axis. In the embodiment shown, the head itself has a diameter greater than the diameter of the screw holes. Washers or other hardware may be used with the fasteners 74, 76 to secure the upper and lower portions 22, 24 to the bone.

A pair of the artificial intervertebral joints 20 may be installed between the vertebrae 14, 16 using a variety of techniques, including one or more of those techniques from the patent applications listed above that have been incorporated by reference. Generally, the artificial intervertebral prosthetic devices 20 may be implanted into a body using a posterior transforaminal approach similar to the known transforaminal lumbar interbody fusion (TLIF) or posterior lumbar interbody fusion (PLIF) procedures. PLIF approaches are generally more medial and rely on more retraction of the traversing root and dura to access the vertebral interspace. TLIF approaches are typically more oblique, requiring less retraction of the exiting root, and less epidural bleeding with less retraction of the traversing structures. It is also possible to access the interspace using a far lateral approach. In some instances it is possible to access the interspace via the far lateral without resecting the facets. Furthermore, a direct lateral approach is known. This approach avoids the posterior neural elements completely. It is anticipated that embodiments of the prosthetic devices 20 could utilize any of these common approaches.

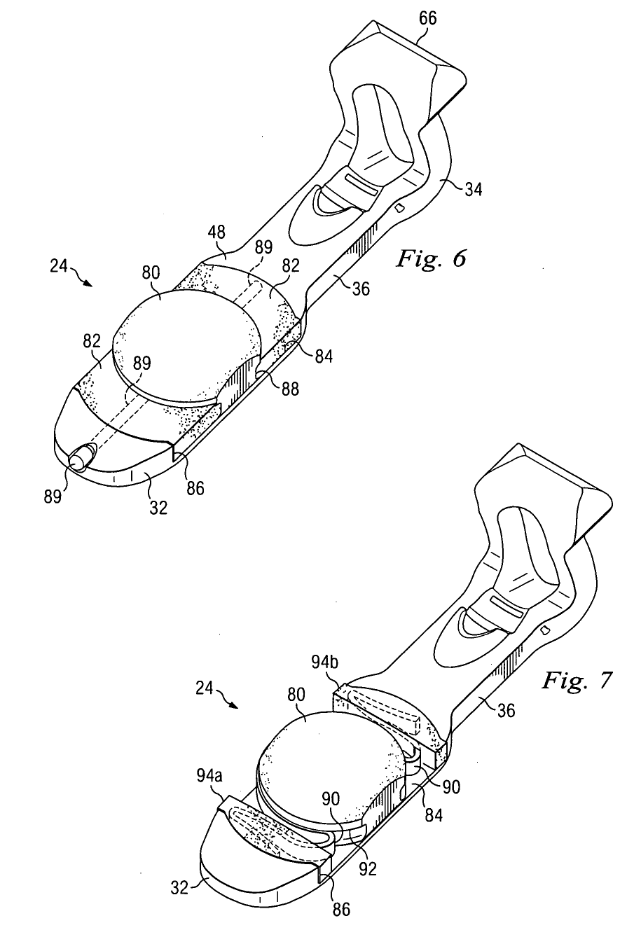

Referring now to FIG. 6, in one embodiment, the articular surface of the lower articular portion 24 is formed on a ball joint 80. The ball joint 80 can be made of the same or different material as the interdiscal section 32. However, in the present embodiment, the ball joint 80 is not rigidly fixed to the interdiscal section 32. Instead, the ball joint 80 is flexibly connected to the interdiscal section 32 by a flexible bumper 82. The flexible bumper 82 is attached to both the ball joint 80 and the interdiscal section 32, such as through chemical and/or mechanical means, but the flexible bumper 82 allows relative movement between the two, especially along a plane parallel with the inner surface 48. The flexible bumper can be made of various materials, including bio-compatible polyurethane and silicon. Although FIG. 6 illustrates the flexible bumper being positioned in a crevice or indention 84 formed in the interdiscal section 32 of the lower articular portion 24, in other embodiment, no such crevice may exist. Furthermore, in some embodiments, the crevice 84 and/or the ball joint 80 may include tapered side walls 86, 88, respectively, similar to a dove-tail, that facilitate the securement of the flexible bumper 82. In some embodiments, the side walls 86, 88 can have different shapes, such as tapered for side wall 86 and “C”-shaped for side wall 88.

In addition to allowing relative movement between the ball joint 80 and the interdiscal section 32, the flexible bumper 82 helps to reduce and/or contain any wear debris that may be caused by the movement. Also, the flexible bumper 82 smoothes the motion by both the flexible nature of the bumper, as well as the ability of the bumper to position a center of rotation (between the two articular portions 22, 24) in a more natural portion of the space S (FIG. 2).

In some embodiments, a cable 89 extends through the interdiscal section 32, the crevice 84, the flexible bumper 82, and the ball joint 80. The cable 89 can be made of metal or other material, including flexible materials. The cable 89 can be secured to the interdiscal section 32 on opposing sides of the crevice 84. The cable 89 can serve to position the ball joint 80 during the manufacture of the flexible bumper 82, and/or can be used to provide extra strength/security to the movement of the ball joint 80 to prevent movement beyond a predefined maximum.

Referring now to FIG. 7, in another embodiment, the ball joint 80 is flexibly connected to the interdiscal section 32 by a spring 90. In the present embodiment, the spring 90 is attached to the ball joint 80 by being positioned under an upper lip 92 of the ball joint. In other embodiments, the spring 90 can be chemically or mechanically fastened or secured around the ball joint, and an additional lower lip or other protrusion of the ball joint can be provided. The spring 90 is positioned inside the crevice 84 to allow relative movement between the ball joint 80 and the interdiscal section 32, especially along a plane parallel with the inner surface 48.

The spring 90 is further connected to the interdiscal section 32 through one or more mechanisms. In the present embodiment, two portions of flexible material 94a, 94b are provided to secure the spring 90 to the side walls 86 of the interdiscal section 32. The flexible material 94a, 94b can be made of various materials, including bio-compatible polyurethane and silicon. The material 94a, 94b can provide benefits similar to those discussed above with reference to the flexible bumper 82 of FIG. 6. In other embodiments, the spring 90 is connected to the interdiscal section through other frictional fit, provided by the expansion tendency of the spring. A sleeve can be provided to cover some or all of the spring 90, as desired.

Referring now to FIGS. 8 and 9, another embodiment of the artificial intervertebral joints 20 discussed above is designated with the reference numeral 120. The artificial intervertebral joint 120 is similar to one or more of the above-described embodiments of the artificial intervertebral joint 20, with differences described in greater detail below.

In one embodiment, the prosthetic device 120 includes an upper articular portion 122 and a lower articular portion 124. The upper articular portion 122 includes an upper main body formed of an interdiscal section 126, a posterior section 128, and a bridge 130 extending between the interdiscal and posterior sections 126, 128. Similarly, the lower articular portion 124 includes a lower main body formed of an interdiscal section 132, a posterior section 134, and a bridge 136 extending between the interdiscal and posterior sections 132, 134.

The upper and lower articular portions 122, 124 may be formed of any suitable biocompatible material, such as those discussed above with reference to articular portions 22, 24, and may further include additional similar features such as screw holes, bone contacting surfaces, and bone connectors.

Together, the first and second articular surfaces may form an articulating joint that allows the upper and lower articular portions 122, 124 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere 180 that may rotate or translate within the first articular surface, forming a loosely constrained ball and socket style joint. In the present embodiment, the sphere 180 is similar to one of the ball joints 80 (and surrounding mechanisms) discussed above with reference to FIGS. 3-7. In other embodiments, the sphere 180 may be a fixed sphere, a resilient disc, or other suitable articulation member.

The posterior sections 128, 134 are disposed at the end of the bridge sections 130, 136 and, in some embodiments, are configured to fit adjacent to the processes of the vertebrae 14, 16. A shock absorber system 140 is positioned between the posterior section 134 of the lower articular portion 124 and the posterior section 128 of the upper articular portion 122. In the present embodiment, the lower articular portion 124 includes a tail 142 extending generally in a direction along the spinal column and the shock absorber system 140 is configured on the tail. Extending upwardly, the tail 142 may be at least partially disposed at a location higher than the bridge section 136. The shock absorber system 140 is attached or positioned around the tail 142, as discussed below.

The shock absorber system 140 may include one or more devices that are responsive to relative movement in different directions between the posterior section 134 and the posterior section 128. In the present embodiment, the shock absorber system 140 includes a coil spring 144, a compression spring 146, a ring 150, and an inner bearing 152.

The coil spring 144 wraps around the tail 142 and rests on the posterior section 128. As compared to some of the other devices of the shock absorber system 140, the coil spring extends a comparatively short distance up the tail 142. The coil spring 144 is provided to absorb side-to-side, or lateral forces, e.g., those forces that are parallel to a plane parallel to the inner surface of the interdiscal section 124. These forces may be provided in response to spinal deformation and/or translational movements between the two vertebrae 12, 14 (FIG. 2). The coil spring 144 may be constructed of a metal, and may include an outer coating that provides additional protection for the spinal environment.

The compression spring 146 also wraps around the tail 142 and rests on the posterior section 128. In the present embodiment, the compression spring 146 is positioned inside the coil spring 144, although in other embodiments, this may be different. As compared to the coil spring 144, the compression spring 146 extends a comparatively high distance up the tail 142. The compression spring 146 is provided to absorb compression and/or decompression forces, e.g., those forces that are perpendicular to the plane parallel to the inner surface of the interdiscal section 124. These forces may be provided in response to spinal flexion and/or extension between the two vertebrae 12, 14 (FIG. 2). The compression spring 146 may be constructed of a metal, and may include an outer coating that provides additional protection for the spinal environment.

The ring 150 also wraps around the tail 142, and in the present embodiment, is positioned between the coil spring 144 and the compression spring 146 and is about the same height as the compression spring. The ring 150 is provided to isolate the coil spring 144 and the compression spring 146. In addition or in the alternative, the ring 150 is provided to support and dampen various forces in any of the above-described directions. In some embodiments, the ring 150 may be used in lieu of the compression spring 146 and/or the coil spring 144. The ring 150 may be constructed of a metal such as aluminum, and may include an outer coating such as Teflon, which is a trademark identifying a product from the DuPont company.

The inner bearing 152 also wraps around the tail 142 and rests on the posterior section 128. In the present embodiment, the inner bearing 152 supports the movement of the coil spring 144, the compression spring 146, and the ring 150. The inner bearing 152 may be constructed of a metal such as aluminum, and may include an outer coating such as Teflon.

In operation, the shock absorber system 144 also fits inside a housing 160 formed in the posterior section 128 of the upper articular portion 122. In the present embodiment, the housing 160 contacts a top portion of the compression spring 146 and/or the ring 150. In some embodiments, the housing 160 may further contact the sides of the coil spring 144. In combination, the two posterior sections 128 and 134 can move in various directions relative to each other, such movement being affected by and/or constrained, at least in part, by one or more components of the shock absorber system 140.

Referring now to FIGS. 10-13, another embodiment of the artificial intervertebral joints 20 and 120 discussed above is designated with the reference numeral 220. The artificial intervertebral joint 220 is similar to one or more of the above-described embodiments of the artificial intervertebral joint 20 and 120, with differences described in greater detail below.

In one embodiment, the prosthetic device 220 includes an upper articular portion 222 and a lower articular portion 224. The upper articular portion 222 includes an upper main body formed of an interdiscal section 226, a posterior section 228, and a bridge 230 extending between the interdiscal portion 226 and posterior section 228. Similarly, the lower articular portion 224 includes a lower main body formed of an interdiscal section 232, a posterior movement section 234, and a bridge 236 extending between the interdiscal and posterior sections 232, 234.

The upper and lower articular portions 222, 224 may be formed of any suitable biocompatible material, such as those discussed above with reference to articular portions 22, 24, and may further include additional similar features such as screw holes, bone contacting surfaces, and bone connectors.

Together, the first and second articular surfaces may form an articulating joint that allows the upper and lower articular portions 222, 224 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere 280 that may rotate or translate within the first articular surface, forming a loosely constrained ball and socket style joint. In the present embodiment, the sphere 280 is similar to one of the ball joints 80 (and surrounding mechanisms) discussed above with reference to FIGS. 3-7. In other embodiments, the sphere 280 may be a fixed sphere, a resilient disc, or other suitable articulation member.

The posterior sections 228, 234 are disposed at the end of the bridge sections 230, 236, respectively, and, in some embodiments, are configured to fit adjacent to the processes of the vertebrae 24, 26. The posterior movement section 234, in the present embodiment, includes one or more flexible components that allow relative movement between the two posterior sections 228, 234 in various directions. In one embodiment, the posterior movement section 234 includes a flexible bumper 242 positioned around a flexible member 244, a post 246, and a flexible coupler 248.

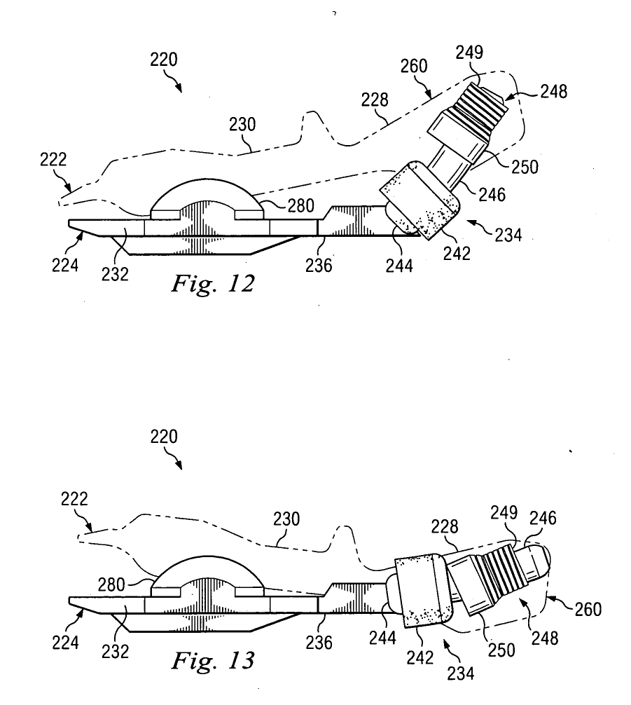

The flexible member 244 is secured to the bridge 236 to allow movement in various directions. FIGS. 12 and 13 illustrate movement in two directions caused by a flexion and extension movement between the two vertebrae 12, 14 (FIG. 2), respectively. The flexible member 244 can be made of a single material or a combination of materials. For example, a portion of the flexible member that connects to the bridge 236 may be formed of a more flexible material, while a portion of the flexible member that extends away from the bridge can be formed of a less flexible material.

The flexible bumper 242 surrounds the flexible member 244, and is provided to absorb dynamic forces in a manner that distributes a load caused by such forces accordingly. The flexible bumper 242 can further be configured to urge the flexible member 244 to a desired or “normal” position (e.g., no flexion or extension). The flexible bumper 242 can be formed of materials such as any rubber or elastic materials.

The post 246 is connected to the flexible bumper 242 and/or the flexible member 244 and extends in a direction away from the bridge 236. The connection can either be rigid, or allow a certain degree of flexibility. The post can be made of a relatively stiff material, such as metal or PEEK. In the present embodiment, the post 246 is constructed of such a length so as to extend into the posterior section 228 of the upper articular portion 222, as discussed in greater detail below.

The flexible coupler 248 is positioned around the post 246. In the present embodiment, the flexible coupler 248 includes a deformable section 249 and a base section 250. The base section 250 is shaped to contact the posterior section 228 of the upper articular portion 222, as discussed in greater detail below. The deformable section 249 may also contact the posterior section 228, depending on the given amount of flexion or extension. The flexible coupler 248 is also configured to allow the post 246 to extend longitudinally through a center portion of the flexible coupler to thereby allow movement between the two posterior sections of the prosthetic device 220. The flexible coupler can be made of metal, with the base section 250 being relatively solid and the deformable section 249 having coil-shaped incisions to support deformation.

In operation, at least a portion of the posterior movement section 234 fits inside a housing 260 formed in the posterior section 228 of the upper articular portion 222. In the present embodiment, the housing 260 includes an opening 262 through which the post 246 can extend to different amounts (depending on the amount of flexion/extension). As shown in FIGS. 12 and 13, the housing 260 contacts the flexible coupler 248 at the base section 250. This contact can move and/or change, depending on the amount of flexion/extension, translation, or other movement between the two vertebrae 12, 14 (FIG. 2). During such movement, one or more components of the posterior movement section 234 can move, bend, or change in various directions relative to each other, such movement being affected by and/or constrained, at least in part, by one or more components of the posterior movement section 234.

Referring now to FIGS. 14-16, another embodiment of the artificial intervertebral joints 20, 120 and 220 discussed above is designated with the reference numeral 320. The artificial intervertebral joint 320 is similar to one or more of the above-described embodiments of the artificial intervertebral joints 20, 120 and 220, with differences described in greater detail below.

In one embodiment, the prosthetic device 320 includes an upper articular portion 322 and a lower articular portion 324. The upper articular portion 322 includes an upper main body formed of an interdiscal section 326, a posterior section 328, and a bridge 330 extending between the interdiscal portion 326 and posterior section 328. Similarly, the lower articular portion 324 includes a lower main body formed of an interdiscal section 332, a posterior section 334, and a bridge 336 extending between the interdiscal and posterior sections 332, 334.

The upper and lower articular portions 322, 324 may be formed of any suitable biocompatible material, such as those discussed above with reference to articular portions 22, 24 (FIGS. 3-7), and may further include additional similar features such as screw holes, bone contacting surfaces, and bone connectors.

Together, the first and second articular surfaces may form an articulating joint that allows the upper and lower articular portions 322, 324 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere 380 that may rotate or translate within the first articular surface, forming a loosely constrained ball and socket style joint. In the present embodiment, the sphere 380 is similar to one of the ball joints 80 (and surrounding mechanisms) discussed above with reference to FIGS. 3-7. In other embodiments, the sphere 380 may be a fixed sphere, a resilient disc, or other suitable articulation member.

The posterior sections 328, 334 are disposed at the end of the bridge sections 330, 336 and, in some embodiments, are configured to fit adjacent to the processes of the vertebrae 14, 16 (FIG. 2). The posterior section 328, in the present embodiment, includes one or more flexible components that allow relative movement between the two posterior sections 328, 334 in various directions. In one embodiment, the posterior section 328 includes a flexible member 342 that engages with the posterior section 334.

The flexible member 342 is positioned and secured inside an opening 344 of the upper posterior section 328. The flexible member 342 is further secured to the posterior section 334. The flexible member 342 can be made of a single material or several different materials. The flexible member 342, in one embodiment, is an elastic rubber membrane that can be attached mechanically and/or chemically to both the posterior sections 328, 334. In another embodiment, the flexible member 342 is only attached to the upper posterior section 328, and the lower posterior section 334 is allowed to move inside the flexible member. Also in some embodiments, the bridge 336 and the posterior section 334 may be made of a different material than the interdiscal portion 332. For example, the bridge 336 and the posterior section 334 may be made of PEEK or some material that supports an amount of movement between the posterior section and the interdiscal portion 332.

Referring specifically to FIGS. 14 and 15, in operation, relative movement between the upper and lower articular portions 322, 324 in two directions 350, 352 (e.g., a flexion or extension movement between the two vertebrae 12, 14 of FIG. 2) is supported by the flexible member 342. The flexible membrane 342 surrounds the posterior section 334, and is provided to absorb dynamic forces in a manner that distributes a load caused by such forces accordingly. The flexible membrane 342 can further be configured to urge the lower articular portion 324 to a desired or “normal” position (e.g., no flexion or extension).

Referring specifically to FIG. 16, in further operation, relative movement between the upper and lower articular portions 322, 324 in two additional directions 354, 356 (e.g., a translational movement between the two vertebrae 12, 14 of FIG. 2) can also be supported by the flexible member 342. The flexible member 344, in the present embodiment, is elastic and therefore can provide a force to return the upper and lower articular portions 322, 324 to a normal (e.g., non-translated) position. In other embodiments, the bridge 336 is able to slide through the flexible member 344 to support the relative movements 354, 356.

It is understood that in various embodiments, one or more of the movement directions 350, 352, 354, and 356 may not be supported. Also, in additional embodiments, further movement directions can be supported, which can allow the prosthetic device 320 to be inserted in a first position by a surgeon, and subsequently allow the lower articular portions 322, 324 to move to a more natural or predetermined position for articulation.

Referring now to FIGS. 17 and 18, another embodiment of the artificial intervertebral joints 20, 120, 220, and 320 discussed above is designated with the reference numeral 420. The artificial intervertebral joint 420 is similar to the artificial intervertebral joints 20, 120, 220, and 320, with differences described in greater detail below.

In one embodiment, the prosthetic device 420 includes an upper articular portion 422 and a lower articular portion 424. The upper articular portion 422 includes an upper main body formed of an interdiscal section 426, a posterior section 428, and a bridge 430 extending between the interdiscal portion 426 and posterior section 428. Similarly, the lower articular portion 424 includes a lower main body formed of an interdiscal section 432, a posterior movement section 434, and a bridge 436 extending between the interdiscal and posterior sections 432, 434.

The upper and lower articular portions 422, 424 may be formed of any suitable biocompatible material, such as those discussed above with reference to articular portions 22, 24 (FIGS. 3-7), and may further include additional similar features such as screw holes, bone contacting surfaces, and bone connectors.

Together, the first and second articular surfaces may form an articulating joint that allows the upper and lower articular portions 422, 424 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere 480 that may rotate or translate within the first articular surface, forming a loosely constrained ball and socket style joint. In the present embodiment, the sphere 480 is similar to one of the ball joints 80 (and surrounding mechanisms) discussed above with reference to FIGS. 3-7. In other embodiments, the sphere 480 may be a fixed sphere, a resilient disc, or other suitable articulation member.

The posterior sections 428, 434 are disposed at the end of the bridge sections 430, 436 and, in some embodiments, are configured to fit adjacent to the processes of the vertebrae 14, 16 (FIG. 2). The posterior section 434, in the present embodiment, includes one or more flexible components that allow relative movement between the two posterior sections 428, 434 in various directions. In one embodiment, the posterior movement section 434 includes an upper flexible bumper 442 and a lower flexible bumper 444.

The flexible bumpers 442, 444 form an opening through which the upper posterior section 428 is positioned and secured. Each of the flexible bumpers 442, 444 can be made of a single material or several different materials. In one embodiment, the flexible bumpers 442, 444 are formed of an elastic rubber material that can be attached mechanically and/or chemically to the lower posterior section 434. Also in some embodiments, the bridge 430 and the posterior section 428 may be made of a different material than the interdiscal portion 426. For example, the bridge 436 and the posterior section 434 may be made of PEEK or some material that supports an amount of movement between the posterior section and the interdiscal portion 432.

Referring specifically to FIG. 17, in operation, relative movement between the upper and lower articular portions 422, 424 in two directions 450, 452 (e.g., a flexion or extension movement) is supported by the flexible bumpers 442, 444. The flexible bumpers 442, 444 surround the posterior section 434, and are provided to absorb dynamic forces in a manner that distributes a load caused by such forces accordingly. The flexible bumper 442 can further be configured to urge the posterior section 434 to a desired or “normal” position (e.g., no flexion or extension). The flexible bumpers 442, 444 can be formed of materials such as any rubber or elastic materials.

Referring specifically to FIG. 18, in further operation, relative movement between the upper and lower articular portions 422, 424 in two additional directions 454, 456 (e.g., a translational movement) can also be supported by the flexible bumpers 442, 444. In some embodiments, an opening 460 is formed in the posterior section 434 through which a portion of the posterior section 428 and/or the flexible bumpers 442, 444 can extend. The flexible bumpers 442, 444, in the present embodiment, are elastic and therefore can provide a force to return the upper and lower articular portions 422, 424 to a normal (e.g., non-translated) position. In other embodiments, the bridge 436 is able to slide through the flexible bumpers 442, 444 to support the relative movements 454, 456. It is understood that in various embodiments, one or more of the movement directions 450, 452, 454, and 456 may not be supported.

Referring now to FIGS. 19 and 20, another embodiment of the artificial intervertebral joints 20, 120, 220, 320, and 420 discussed above is designated with the reference numeral 520. The artificial intervertebral joint 520 is similar to the artificial intervertebral joints 20, 120, 220, 320, and 420, with differences described in greater detail below.

In one embodiment, the prosthetic device 520 includes an upper articular portion 522 and a lower articular portion 524. The upper articular portion 522 includes an upper main body formed of an interdiscal section 526, a posterior section 528, and a bridge 530 extending between the interdiscal portion 526 and posterior section 528. Similarly, the lower articular portion 524 includes a lower main body formed of an interdiscal section 532, a posterior section 534, and a bridge 536 extending between the interdiscal and posterior sections 532, 534.

The upper and lower articular portions 522, 524 may be formed of any suitable biocompatible material, such as those discussed above with reference to articular portions 22, 24 (FIGS. 3-7), and may further include additional similar features such as screw holes, bone contacting surfaces, and bone connectors.

Together, the first and second articular surfaces may form an articulating joint that allows the upper and lower articular portions 522, 524 to articulate relative to each other. This articulation, in turn, may allow articulating movement of the upper vertebra 14 relative to the lower vertebra 16, and in some embodiments, may allow movement similar to that provided by a natural spinal disc. In the embodiment shown, the second articular surface is a partial sphere 580 that may rotate or translate within the first articular surface, forming a loosely constrained ball and socket style joint. In the present embodiment, the sphere 580 is similar to the ball joint 80 discussed above with reference to FIG. 6 and is attached to a flexible bumper 582, which is further attached to the interdiscal section 532, to allow movement there-between.

The posterior sections 528, 534 are disposed at the end of the bridge sections 530, 536 and inside a movement device 540. The movement device 540 includes a housing 542, a sliding member 544, and a hinge 546. In the present embodiment, the sliding member 544 and hinge 546 are made of metal and the housing 542 is made of PEEK. In other embodiments, one or more of the components of the movement device 540 may be made of a flexible material to support various types of movement between the upper and lower articular portions 522, 524.

In the present embodiment, the lower posterior section 534 is configured to fit adjacent to the articulating process of the inferior vertebrae 16 (FIG. 2), and the housing 542 is configured to fit adjacent to the articulating process of the superior vertebrate 14. As such, one or both of the lower posterior section 534 and housing 542 may include various components (e.g., tethers) and or surfaces to facilitate the connection and interaction with the respective spinous process.

The upper posterior section 528 includes an opening through which the sliding member 544 can move. The lower posterior section 534 also includes an opening which is secured to the sliding member 544. The sliding member 544 is further secured to a superior portion of the housing 542.

In operation, relative movement between the upper and lower articular portions 522, 524 in two directions 550, 552 (e.g., a flexion or extension movement between the two vertebrae 12, 14 of FIG. 2) is supported by the movement device 540. As shown in FIGS. 19 and 20, an amount of flexion and extension, respectively, can occur by the upper posterior section 528 moving along the sliding member 544. It is understood that in some embodiments, the sliding member 544 can be arched to support the flexion/extension relative movement between the two posterior sections 528, 534. Also in some embodiments, the sliding member 544 can have a different shape or contour near the ends (where full flexion or extension would occur). Even further, flexible members can be provided to help absorb shock between the two posterior sections 528, 534 and encourage the sections into a more normal position.

Referring now to FIGS. 21-24, another embodiment of the artificial intervertebral joints 20, 120, 220, 320, 420, and 520 discussed above is designated with the reference numeral 620. The artificial intervertebral joint 620 is similar to the artificial intervertebral joints 20, 120, 220, 320, 420, and 520, with differences described in greater detail below.

In one embodiment, the prosthetic device 620 includes an upper articular portion 622 and a lower articular portion 624. The upper articular portion 622 includes an interdiscal section 626 and a posterior section 628, without a bridge extending there-between. The lower articular portion 624 includes a lower main body formed of an interdiscal section 632, a posterior section 634, and a bridge 636 extending between the interdiscal and posterior sections 632, 634.

The upper and lower articular portions 622, 624 may be formed of any suitable biocompatible material, such as those discussed above with reference to articular portions 22, 24 (FIGS. 3-7), and may further include additional similar features such as screw holes, bone contacting surfaces, and bone connectors.

Together, the first and second articular surfaces may form an articulating joint that allows the upper and lower articular portions 622, 624 to articulate relative to each other. This articulation, in turn, may allow articulating movement of the upper vertebra 14 relative to the lower vertebra 16, and in some embodiments, may allow movement similar to that provided by a natural spinal disc.

In the embodiment shown in FIGS. 21-23, the lower articular portion 624 includes a fulcrum 680 about which the upper articular portion 622 can move. In the present embodiment, the fulcrum 680 is attached to a flexible bumper 682, which is further attached to the interdiscal section 632, to allow movement there-between. In some embodiments, the fulcrum 680 may further include a pin or shaft 684 for allowing the articulation between the upper and lower articular portions 622, 624, while securely maintaining the two articular portions in a predetermined position. This can facilitate insertions of the prosthetic device 620 and allows the upper articular portion 622 to be secured without using additional means (such as a bone screw).

In the embodiment shown in FIG. 24, the second articular surface is a partial sphere 681 that may rotate or translate within the first articular surface, forming a loosely constrained ball and socket style joint. In the present embodiment, the sphere 580 is similar to the ball joint 80 discussed above with reference to FIG. 6 and is attached to a flexible bumper 682, which is further attached to the interdiscal section 632, to allow movement there-between.

The posterior section 634 is disposed at the end of the bridge section 636; the posterior section 628 is not directly connected to the articular portion 626. Both posterior sections 628, 634 are further positioned inside a movement device 640. The movement device 640 includes housings 642, 644 (which are connected in the embodiment of FIG. 21 and separate in the embodiment of FIG. 22) and hinges 646, 648. In the present embodiment, the hinges 646, 648 are made of metal and the housings 642, 644 are made of PEEK. In other embodiments, one or more of the components of the movement device 640 may be made of a flexible material to support various types of movement between the upper and lower articular portions 622, 624. In the present embodiment, the lower posterior section 634 is configured to fit adjacent to the articulating process of the inferior vertebrae 16 (FIG. 2).

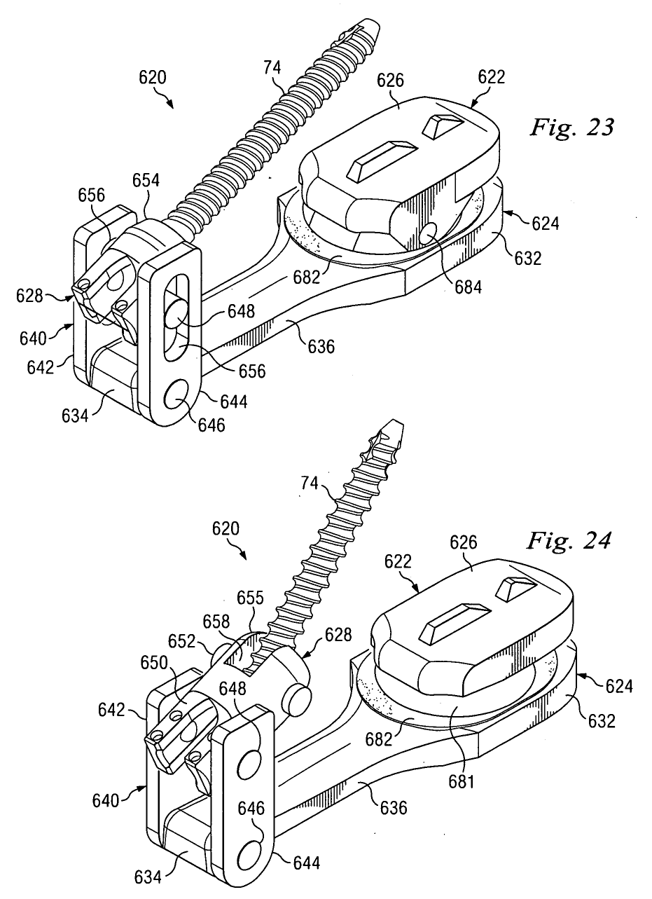

The housings 642, 644 are rotatably connected to the lower posterior section 634 through the hinge 646. The housings 642, 644 are also rotatably connected to the upper posterior section 628 through the hinge 648. The upper posterior section 628 further includes a retaining member 650 with a fixed holder 654 for receiving and securing to a fastener 74 such as a bone screw. The bone screw 74 can be a standard pedicle screw (e.g., for the embodiments of FIGS. 23 and 24), a multi-axial screw (e.g., for the embodiments of FIGS. 21 and 22), or other types of screws. In the embodiment of FIG. 21, the housings 642, 644 are connected by a bridge portion 659, while in the embodiments of FIGS. 22-24, there is no bridge portion.

In the embodiment of FIG. 22, the retaining member 650 further includes a fixation joint 652 and a holder 655 that are movable relative to the portion of the retaining member that connects to the hinge 648. This allows the bone screw 74 to be inserted into and fixed to the superior vertebrae 14 (FIG. 2) at various angles. In one embodiment, the fixation joint 652 becomes secured when the bone screw 74 is tightened to the vertebrae. In the present embodiment, the fixation joint 652 retains the ability to move, even after the bone screw 74 has been tightened to the vertebrae.

In the embodiment of FIG. 23, a slot 656 is provided in the housings 642 and 644 through which the hinge 648 can slide. This allows the holder 654 to move up and down inside the housings, facilitating the positioning of the bone screw 74 and supporting movement between the opposing vertebrae.

In the embodiment of FIG. 24, the fixation joint 652 provides relative movement between the bone screw 74 and the holder 655. Also, the holder 655 includes a slot 658 through which the bone screw 74 can move. This also allows the bone screw 74 to be inserted into and fixed to the superior vertebrae 14 (FIG. 2) at various angles. In one embodiment, the fixation joint 652 becomes secured when the bone screw 74 is tightened to the vertebrae. In the present embodiment, the fixation joint 652 retains the ability to move, even after the bone screw 74 has been tightened to the vertebrae.

In operation, relative movement between the upper and lower posterior sections 628, 634 is allowed in multiple directions by the hinges 646, 648 (and for some embodiments, the fixation joint 652). Translational movement can be supported, in part, by the hinges 646 and 648. Flexion and extension can be supported, in part, by the hinge 648 and the fixation joint 652.

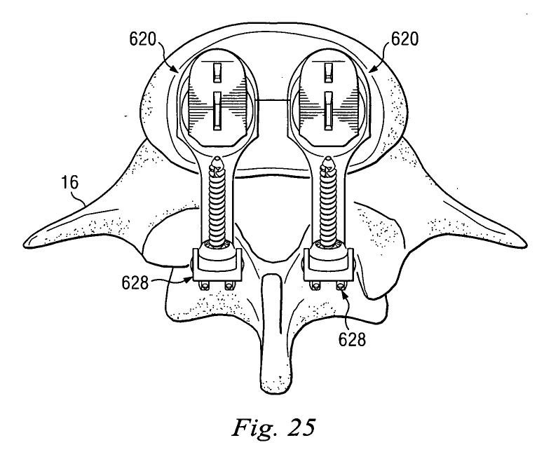

All of the embodiments provide many benefits over those of the prior art. Many of the embodiments can be inserted using different surgical techniques. For example, as shown in FIG. 25, a pair of the artificial intervertebral joints 620 can be inserted at various approaches, including a straight posterior approach, as an alternative to the approaches discussed above with reference to FIG. 4. In this embodiment, the posterior sections 628, 634 do not have to be attached to spinous processes, but can attached to other portions of the posterior arch, including the lamina. Also, the intervertebral joints can be relatively stable and self-centering. Both the anterior joint and the posterior connection allow the prosthetic device to resist shear forces, particularly anterior-posterior forces.

The robust and forgiving structure of the anterior joint permits misalignment and slight inaccuracy in the placement of the prosthetic devices. For example, the ball and socket structure of the articular joint tolerates a certain amount of misalignment between the components. As such, certain embodiments of the prosthetic device, in accordance with the present disclosure, may utilize the interdiscal sections alone, without direct connection to a separate posterior section, with a different kind of posterior implant, or with no posterior implants at all.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “superior,” “inferior,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

We claim:

1. A prosthetic device for placement in an intervertebral space defined between a first vertebra and a second vertebra to provide articulating motion to the first and second vertebrae, comprising:
a first articular portion configured to be at least partially disposed in the intervertebral space and adjacent to the first vertebra;
a second articular portion configured to be at least partially disposed in the intervertebral space and adjacent to the second vertebra;
a hemispherical member about which the first and second articular portions can articulate; and
a flexible member for flexibly securing the hemispherical member to the first articular portion.

2. The prosthetic device of claim 1 further comprising:
a first posterior section connected to one of either the first or second articular portions, the first posterior section being disposed at a location posterior of the intervertebral space.

3. The prosthetic device of claim 1, wherein the flexible member includes a rubber bumper for allowing the hemispherical member to move in a plane parallel with a top surface of the first articular portion.

4. The prosthetic device of claim 1, wherein the flexible member includes a spring for allowing the hemispherical member to move in a plane parallel with a top surface of the first articular portion.

5. The prosthetic device of claim 4, wherein the flexible member also includes a rubber bumper.

6. The prosthetic device of claim 5, wherein the flexible member is configured to re-position a center of articulation after the prosthetic device has been placed in the intervertebral space.

7. The prosthetic device of claim 1 wherein:
the first articular portion includes a first posterior section and a first interdiscal section, the first posterior section being configured for connection to an anterior arch of the first vertebra and the first posterior section being configured for connection to a posterior arch of the first vertebra;
the second articular portion includes a second posterior section and a second interdiscal section, the second posterior section being configured for connection to an anterior arch of the second vertebra and the second posterior section being configured for connection to a posterior arch of the second vertebra.

8. The prosthetic device of claim 7 further comprising:
a shock absorber system positioned between the first posterior section and the second posterior section.

9. The prosthetic device of claim 8 wherein the shock absorber system includes a coil spring positioned around a tail of either the first or second posterior section and inside a housing of the other posterior section.

10. The prosthetic device of claim 8 wherein the shock absorber system includes a compression spring positioned between the first and second posterior sections.

11. The prosthetic device of claim 9 wherein the shock absorber system further includes:
a compression spring positioned around the tail and against the housing; and
a bumper between the coil spring and the compression spring.

12. The prosthetic device of claim 7 further comprising:
an opening in one of either the first or second posterior sections; and
a tail connected to the other of either the first or second posterior sections, wherein the tail is extendable through the opening.

13. The prosthetic device of claim 12 further comprising:
a flexible member positioned in the opening, whereby the flexible member is configured to dampen forces exerted between the tail and the opening.

14. The prosthetic device of claim 12 further comprising:
a flexible member positioned in the opening, whereby the flexible member is configured to support constrained movement of the tail in the opening.

15. The prosthetic device of claim 14, wherein the constrained movement includes both flexion and extension.

16. The prosthetic device of claim 14, wherein the constrained movement includes translational movement between the first and second articular portions.

17. The prosthetic device of claim 12 wherein the tail is connected to the respective posterior section by a flexible member.

18. The prosthetic device of claim 17 further comprising a flexible coupler configured adjacent to the tail, whereby the flexible coupler is positioned to engage with the opposite posterior section in a manner to absorb forces between the two posterior sections.

19. The prosthetic device of claim 18 wherein the tail is connected to the respective posterior section by a flexible bumper.

20. The prosthetic device of claim 12 further comprising a flexible coupler around the tail, whereby the flexible coupler is positioned to engage with the opposite posterior section in a manner to constrain flexion and extension between the two posterior sections.

21. The prosthetic device of claim 7, further comprising:
a first bridge section positioned between and securing the first posterior section to the first interdiscal section.

22. The prosthetic device of claim 21, further comprising:
a securement mechanism for securing the second posterior section to the posterior arch of the second vertebra.

23. The prosthetic device of claim 22, wherein the securement mechanism includes an opening for receiving a bone fastener and a slot for allowing movement of the bone fastener.

24. The prosthetic device of claim 22, wherein the securement mechanism includes a fixation joint for securing to the first posterior section.

25. The prosthetic device of claim 24 wherein the fixation joint is flexibly secured to the first posterior section.

26. The prosthetic device of claim 24 wherein the fixation joint is secured to the first posterior section through a movement device.

27. The prosthetic device of claim 24 wherein the movement device includes at least one housing and at least one hinge.

28. The prosthetic device of claim 24 wherein the movement device includes a first hinge for rotatably securing to the fixation joint and a second hinge for rotatably securing to the first posterior section.

29. A motion-preserving prosthetic device component for placement in an intervertebral space defined between a first vertebra and a second vertebra, comprising an intervertebral section configured to be at least partially disposed in the intervertebral space, and a posterior section configured to be at least partially disposed outside of the intervertebral space, wherein the intervertebral section and the posterior section each separately support motion, and wherein the posterior section includes a flexible movement controlling mechanism.

30. The prosthetic device of claim 29, wherein the posterior section includes a spring for controlling relative movement in posterior arches of the first and second vertebrae.

31. The prosthetic device of claim 30, wherein the spring is a coil spring for absorbing side-to-side movements.

32. The prosthetic device of claim 30, wherein the spring is a compression spring for absorbing flexion/extension movements.

33. The prosthetic device of claim 29, wherein the posterior section includes a flexible coupler configured for controlling relative movement in posterior arches of the first and second vertebrae.

34. The prosthetic device of claim 33, wherein the flexible coupler is attached about a post extending between opposing portions of the posterior section.

35. The prosthetic device of claim 34, wherein the post is flexibly attached to at least one of the opposing portions of the posterior section.

36. The prosthetic device of claim 29, wherein the posterior section includes a flexible bumper for controlling relative movement in posterior arches of the first and second vertebrae.

37. The prosthetic device of claim 29, wherein the posterior section includes a hinge for controlling relative movement in posterior arches of the first and second vertebrae.

38. The prosthetic device of claim 29, wherein the posterior section includes a rod about which a portion of the posterior section can slide.

39. The prosthetic device of claim 38, wherein the posterior section includes a housing and the rod is positioned in a slot inside the housing.

40. The prosthetic device of claim 37, wherein the posterior section further includes a fixation joint for rotatably securing the posterior section to one of the two vertebra.

41. A method of implanting a prosthetic device including an interbody component, a posterior component, and a combined interbody/posterior component, the method comprising:
placing the combined interbody/posterior component in an intervertebral space between first and second vertebrae, the combined interbody/posterior component being placed to engage with an anterior arch and a posterior arch of the first vertebra;
placing the interbody component in the intervertebral space, the interbody component being placed to engage with an anterior arch of the second vertebra; and
placing the posterior component in the intervertebral space, the posterior component being placed to engage with a posterior arch of the second vertebra.

42. The method of claim 41, further comprising:
attaching the posterior component to the posterior arch of the second vertebra by introducing a fastener through an aperture in the posterior component.

43. The method of claim 41, further comprising:
attaching the interbody component to the anterior arch of the second vertebra by introducing a keel into an endplate of the second vertebra.

44. The method of claim 41, further comprising:
attaching the combined interbody/posterior component to the first vertebra by introducing a fastener through an aperture in the combined interbody/posterior component and into the first vertebra.
